(19) United States
(12) Patent Application Publication
(10) Pub. No.: US 2002/0103508 A1
(43) Pub. Date: Aug. 1, 2002

(54) Remotely operated defibrillator

(76) Inventor: Prabodh Mathur, Laguna Niguel, CA (US)

Correspondence Address: GOTTLIEB RACKMAN & REISMAN PC, 270 MADISON AVENUE, 8TH FLOOR, NEW YORK, NY, 100160601

(21) Appl. No.: 09/772,017

(22) Filed: Jan. 29, 2001

(51) Int. Cl. A61N 1/39

(52) U.S. Cl. 607/5

(57) ABSTRACT

An external cardiac device includes a detector used to detect an abnormal condition of a patient, a controller operating the defibrillator in response to a command and a therapy delivery circuit that delivers appropriate therapy, such antitachycardia, defibrillation or antibradicardia therapy. The defibrillator is attached to a patient by any attendant or bystander and a signal is sent to central station to alert a clinician that the device has been activated. Once the device has been attached, the defibrillator is adapted to monitor the patient and to transmit information to the remotely located operator. The operator then decides what kind of therapy is required and transmits an appropriate command to the device. In one embodiment, the device includes a base station communicating with the central station and a pulse generator which may be separable from the base station. In another embodiment a unitary pulse generator is used with means for communicating with the clinician directly. The device includes a speaker and a microphone to allow the operator and the attendant and/or patient to communicate with each other.

### BACKGROUND OF THE INVENTION

A. Field of Invention

This invention pertains to an external defibrillator system adapted to provide therapy selectively to patients suffering from sudden acute cardiac arrest. More particularly, the present invention pertains to an external defibrillator system including a pulse generator adapted to apply therapeutic pulses to a patient, a local controller which is normally interfaced with the pulse generator, except during the application of therapy, and a remote controller operated by a clinician which generates commands to control the operation of the pulse generator. The human operator receives instructions on how to position the pulse generator and other information from the clinician via a voice communication link.

2. Description of the Prior Art

The term Cardiac Emergency is used herein to denote situations leading to acute, potentially life threatening symptoms that may be caused by cardiac disease.

The term Sudden Cardiac Arrest or SCA in a patient refers to a condition characterized by a loss of effective pumping action in the heart. More specifically, the patient's heart loses rhythm, starts to quiver and ceases to pump blood. The heart then usually goes into ventricular tachycardia (VT) and then typically generates to ventricular fibrillation (VF).

During a heart attack (also called myocardial infarction) the blood vessels to the heart are clogged and the pumping capacity of the heart gets progressively reduced. If left untreated the heart muscles begin to die. The therapy for this condition typically consists of the administration of thrombolytic ( clot dissolving) drugs administered in an emergency health care facility.

Thus, SCA results in an abrupt cessation of blood circulation to the vital organs, and once it occurs, unless the patient's heart is reverted rapidly to a sinus rhythm, death will occur. In fact SCA is considered to be the leading cause of death in the United States and throughout the world. The acute therapy for VT and VF consists of electrical shocks which are generally characterized as cardioversion pulses and ventricular defibrilliation shocks, respectively.

Arrhythmias which cause SCA include ventricular tachycardia and ventricular fibrillation. Ventricular tachycardia is characterized by electrical disturbances which cause a dangerously high cardiac rate and may lead to ventricular fibrillation. Ventricular fibrillation refers to a state where cardiac electrical activity is completely disorganized and the heart is quivering. During ventricular fibrillation, the heart does not pump blood, and no beats can be detected.

Other arrhythmias can lead to acute symptoms as well, including fainting, dizziness which, if left untreated, could become life threatening. These latter arrhythmias include bradycardia (which occurs when a person's heat beat slows down so much that not enough blood is being pumped through the body) and supra-ventricular arrhythmias (SVT).

Arrhythmias may be detected from the patient's electrocardiogram (ECG), blood pressure, blood oxygenation level and other similar physiological parameters. Because the signals indicative of these parameters can be very complex, various algorithms are used to analyze these parameters to detect and classify an arrhythmia. Once detected, the arrhythmia can be eliminated by using antitachycardia therapy consisting of electrical stimulation. Two kinds of devices are presently available to provide antitachyarrhythmia therapy: internal or implanted cardioverter defibrillators (ICDs), and external defibrillators.

ICDs have been known since the early 1980s. These devices are implanted in the patient and include electrodes extending to the cardiac chambers to sense intrinsic cardiac activity and to provide stimulation signals. The intrinsic signals sensed in the cardiac chambers are used to classify the condition of the heart, and if a tachyarrhythmia is detected, then either cardioversion pacing pulses or defibrillation shocks are applied.

In order for these kinds of devices to function properly, a clinician examines the patient and, after implantation, programs a plurality of parameters into the ICD which are used by a processor to classify the condition of the patient and determine the characteristics of the stimulation signals to be applied. Frequently these parameters are selected after the patient's heart rate is increased either naturally, with exercise, or with drugs. It is advisable to reprogram these parameters as the condition of the patient changes over time.

External defibrillators capable of providing defibrillation shocks or other types of therapy are also well known. In case of a cardiac emergency, current external defibrillators must be operated manually by a trained professional such as an emergency medical technician, paramedic, firefighter, or police officer, etc. Existing external defibrillators do not monitor cardiac activity before a sudden cardiac arrest episode, and accordingly, the professional must examine the patient and determine his condition first, before any therapy is provided. Hence, inherently, the existing external defibrillators cannot be used by a layperson who has not received any training on how to operate the specific external defibrillator.

An external defibrillator, described in commonly assigned U.S. Pat. No. 5,474,574 and incorporated herein by reference, includes an ECG sensor and requires several parameters to be programmed by a clinician before it can be used properly. Some of the programmable parameters pertain to the sensitivity of the ECG sensor required to detect ECGs reliably. Other parameters pertain to the size, number and duration of the shocks to be applied by the device. Since these parameters must be programmed separately for each patient, by the time this defibrillator is ready to be used, it is configured to a specific patient and cannot be used for a different patient without first reprogramming its parameters.

In summary, existing external defibrillators are limited in that they must be operated by a professional, they do not have the capability to continuously monitor a patient and they require active intervention to initiate any therapy.

There is a need for an external defibrillator which can be used successfully in case of a cardiac emergency by a layman, i.e., a person without any formal medical training under the supervision of a remotely located professional clinician.

### OBJECTIVES AND ADVANTAGES OF THE INVENTION

In view of the above, an objective of the present invention is to provide an external defibrillator system which can be distributed and placed at public places which can be used effectively by a person with no special medical training.

A further objective is to provide an external defibrillator system able to allow remote monitoring of a patient and determine automatically if a patient is in need of therapy. A further objective is to provide an external defibrillator system through which a professional clinician from a remote location can provide instructions to an untrained person on how to secure the same to a patient, and then provide cardiac therapy.

Other objectives and advantages of the invention will become apparent from the following description.

Briefly, an external defibrillator system constructed in accordance with this invention includes a pulse generator and a base station adapted to hold the pulse generator when not in use and to provide continuous interfacing between the pulse generator and a trained medical professional. The pulse generator includes a sensing circuit used to sense physiological signals indicative of cardiac activity, a therapy delivery circuit that generates pacing or shock pulses, a controller that is used to operate the defibrillator in response to commands and a transceiver that provides communication directly or indirectly with the professional. Signals indicative of intrinsic cardiac activity, including R-waves and ventricular fibrillation, for example, are detected and transmitted to site of the professional. These signals are then classified as a cardiac condition either by the professional clinician or by an automated expert system. The clinician then generates a command designating a certain therapy to be applied to the patient. The command is sent to the pulse generator which then generates antiarrhythmia pulses appropriate to the patient's cardiac condition. Preferably the therapy is applied synchronously with the intrinsic cardiac activity of the patient.

Preferably, the pulse generator also has a speaker and microphone through which an untrained person can communicate with the professional for the operation of the pulse generator. The pulse generator may also have a display on which some instructions can be provided.

Power for the therapy is provided by rechargeable batteries incorporated into the pulse generator. The battery is then trickle charged while the pulse generator is coupled to the base station.

The base station is provided to hold the pulse generator while not in use, and acts as a repeater station to allow voice and data communication between the pulse generator and the remote location.

### BRIEF DESCRIPTION OF THE DRAWINGS

FIG. 1 shows a block diagram of an external defibrillator system constructed in accordance with this invention;

FIG. 2 shows a block diagram of the pulse unit for the system of FIG. 1;

FIG. 3 shows a block diagram of the base station for the system of FIG. 1;

FIG. 4 shows a block diagram of the central station used to control the system of FIG. 1;

FIG. 5 shows a more detailed diagram of the pulse unit of FIG. 2; and

FIG. 6 shows a flow chart for the operation of the system.

### DETAILED DESCRIPTION OF THE INVENTION

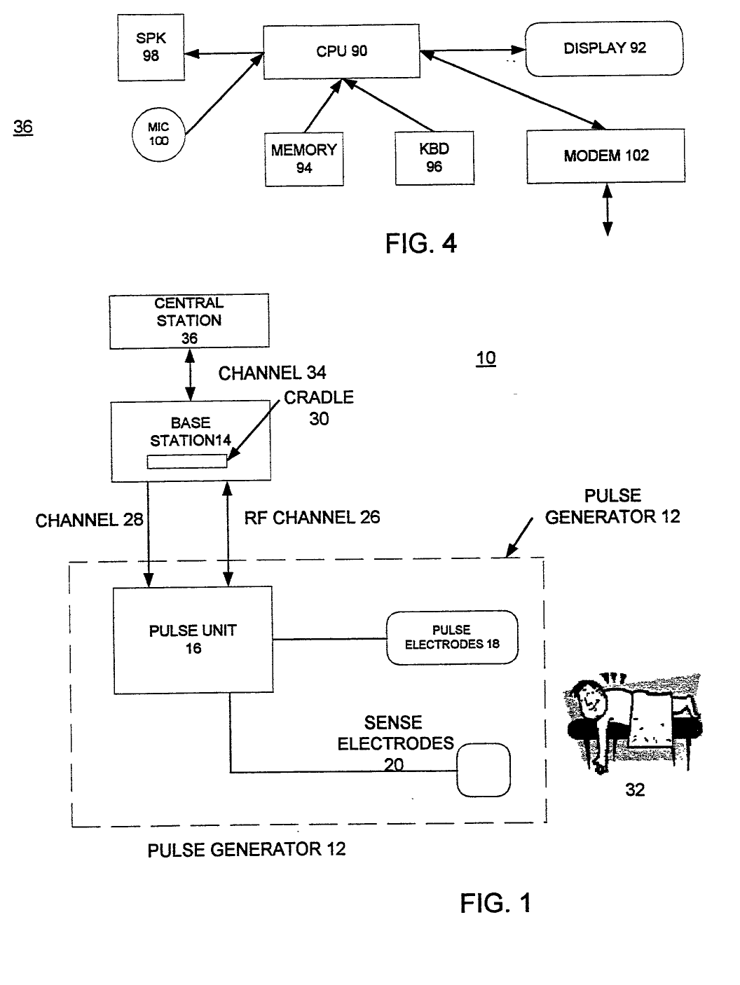

Referring first to FIG. 1, an external defibrillator system 10 constructed in accordance with this invention includes a pulse generator 12 and a base station 14. The pulse generator 12 includes a pulse unit 16 connected to a set of pulse electrodes 18 and a set of sense electrodes 20 by respective cables 22 and 24. Other electrical coupler members may be used instead of the electrodes. Sensing may also be performed through other electrodes as well.

The pulse unit 16 is also coupled to the base station by two channels. The first channel is an RF channel 26 and the second channel is either an inductive or wired channel 28. The channel 28 is used to provide a charging current to rechargeable batteries within the unit 16 (discussed more fully below) and to exchange control signals when the pulse unit 16 is mounted in a cradle 30 of base station 14. Channel 26 is used for exchanging control and voice-grade signals between the unit 16 and base station 14 when the unit 16 is removed from the cradle and used to apply therapy to a patient 32.

The base station 14 is connected by a standard communication channel 34 to a central station 36. The central station 36 is manned by a professional 38 who selectively receives information through pulse generator 12, base station 14 and sense electrodes 20 about patient 32 and who can also send commands to the pulse generator 12 to generate pulses defining a predetermined cardiac therapy. The pulses are applied to the patient 32 by pulse electrodes 18.

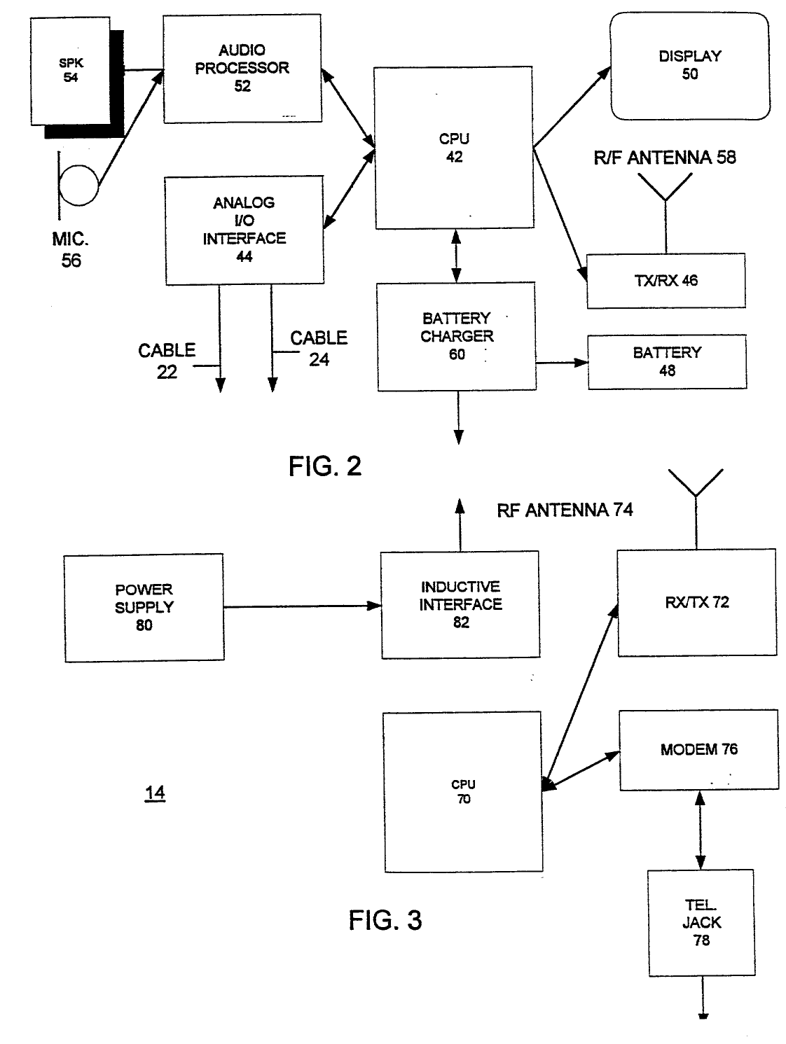

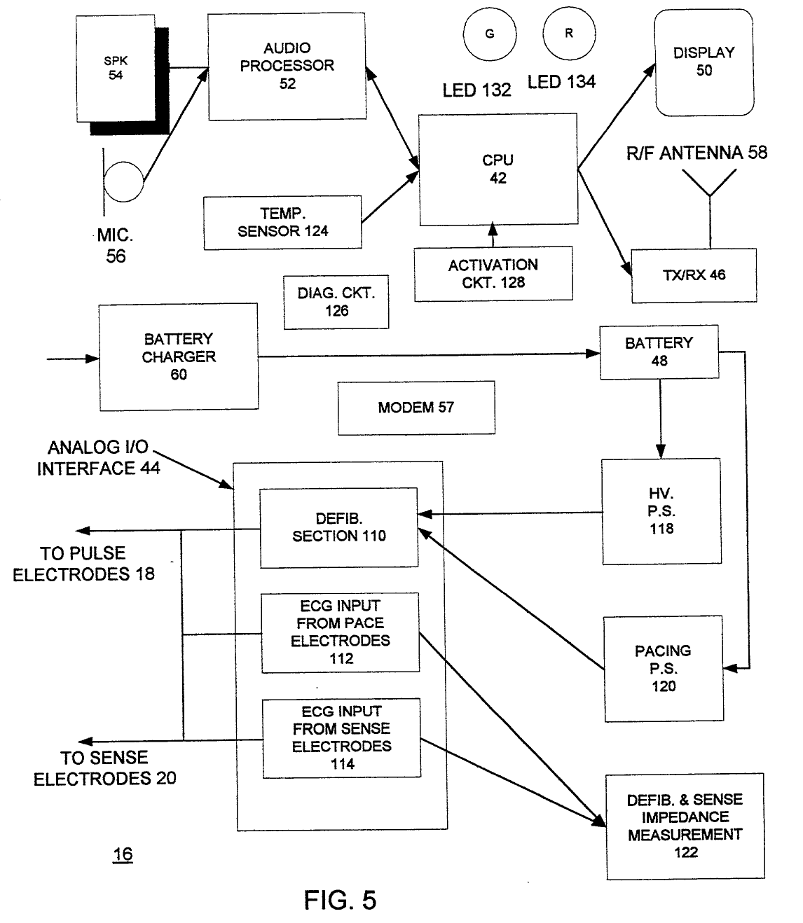

FIG. 2 shows a block diagram of the pulse generator 12. As can be seen in this figure, pulse unit 16 includes a CPU 42, an analog input/output (I/O) interface 44, a transceiver 46 and a rechargeable battery 48. The housing may also include a display 50 and an audio processor 52 associated with a speaker 54 and a microphone 56. The housing 40 may also include an optional modem 57(shown in FIG. 5). The cables 22, 24 are connected to I/O interface 44. The transceiver 46 is coupled to an RF antenna 58. The rechargeable battery 48 provides power to the other circuitry disposed in housing 40 and is coupled to a charging circuit 60. The transceiver 46 and antenna 58 cooperate to maintain communication with the base station 14.

The base station 14, shown in FIG. 3, has its own CPU 70, a transceiver 72 to exchange messages with the pulse unit 16 via an antenna 74 and a modem 76 coupled to a standard telephone jack 78. The base station 14 further includes a power supply 80 which is connected to a standard AC line (not shown) and provides power to an inductive interface 82. The interface 82 provides energy for a trickle charge to the battery 48 via the battery charger circuit 60.

Finally, for the sake of completeness, details of the central station 36 are discussed. Station 36 includes a CPU 90 coupled to a display 92, a memory 94, a keyboard 96, a speaker 98 and a microphone 100. Communication with a plurality of base stations, such as station 14, is established through a modem 102. Obviously, other means of communicating between the central station 36 and base station 14 can be provided as well, such as cellular and other wired and wireless telephone connections, Internet connections and so on.

FIG. 5 shows more details of the pulse unit 16. As it can be seen in this figure, the analog interface 44 may have three sections: a defibrillator section 110, a first ECG section 114 and a second ECG input section 116. Associated with the analog interface 44 is a high voltage power supply 118, a low voltage pacing power supply 120, and a lead impedance measurement circuit 122. The pulse unit 16 also includes an optional temperature sensor 124, a diagnostic circuit 126, an activation circuit 128. The temperature sensor 124 is used to provide an indication of the patient's condition.

Disposed in the housing of the pulse unit (not shown) there is provided the screen 50 and a couple of LEDs, a green LED 132 and a red LED 134 which are used to indicate the status of the defibrillator system, or at least the pulse unit 40.

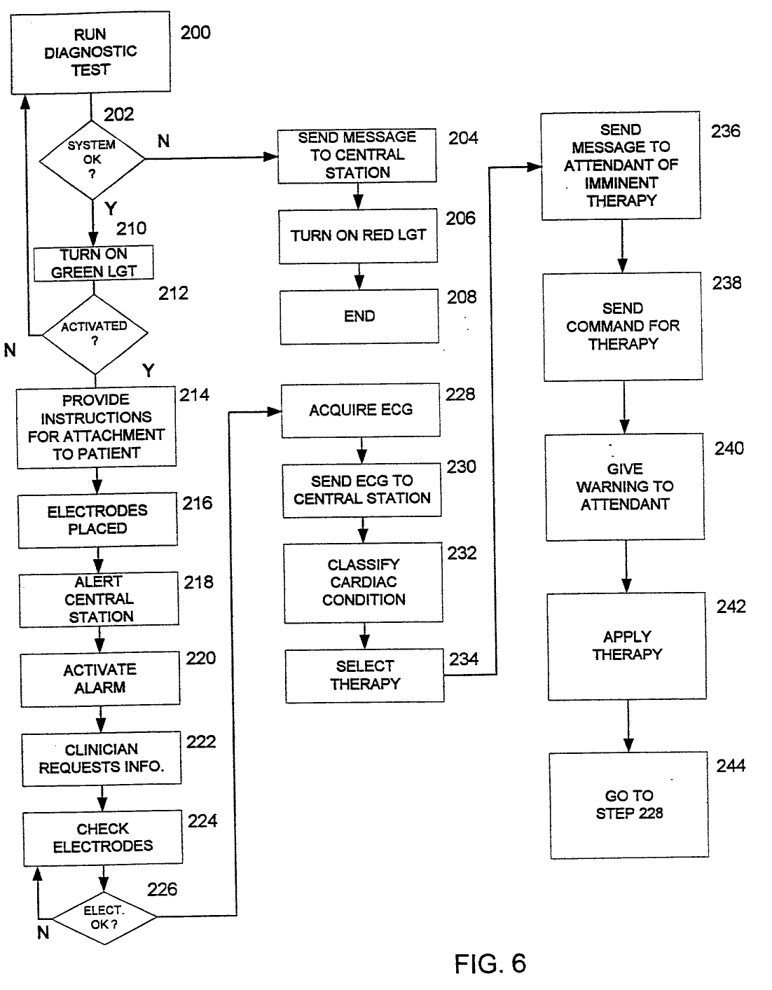

The operation of the defibrillator system is now described in conjunction with the flow chart of FIG. 6 and the other FIGS. 1-5. In step 200 the system runs a diagnostic test of itself using diagnostic circuit 126. This circuit 126 may be adapted to check various functions including the microprocessors 42, 70, the communication link between the pulse unit 16 and base station 14 and between the base station 14 and the central station 36, the charge on batteries 48 and so on. This test is performed at regular intervals, for example, once a second. Alternatively some tests may be run every time while other tests may be run at rarer intervals, for example once a week or once a month. In step 202 at the end of the tests a decision is made as to whether the system 10 is operational or not. If it is not then in step 204 a message is sent to the central station providing an identification code identifying the system 10 and its physical location, and the problems associated with the system 10, if known. Next, in step 204 the RED led 134 is activated and the system then goes into a stand-by mode in step 208 and the process is terminated. The red LED is activated to show that the system 10 is inoperative. A message to this effect may be displayed on screen 50. The problem(s) of the system 10 may also be posed on the display 50.

If in step 202 no system failure is found then in step 210 the green LED is activated to indicate that the system is operational. Next, in step 212 a test is performed to determine if the system has been activated. Although it is preferable to provide the pulse unit 16 with as few manual controls as possible, some such controls may have to be provided. For example, a manual activating circuit 128 may have to be included. The circuit 128 may include a push-button (not shown) mounted on the housing. The purpose of this circuit 128 is to wake the system up and indicate that a cardiac incident is in progress and that the system is required to provide therapy. Of course the circuit 128 may include some automatic elements as well, such as a motion detector(not shown), a proximity detector, etc. In step 212 a check is performed to determine if activating circuit 128 has been triggered. If not, then the system continues in its diagnostic mode.

While in the diagnostic mode, the pulse unit is resting on, or is otherwise coupled to the base station 12 . The power supply 80 in the base station 12 feeds power to the inductive interface 82. The inductive interface 82 then generates an inductive field which is used to excite an inductive power input circuit within the battery charger 60 in the pulse unit 16. The battery charger 60 which provides a trickle charge to battery 48. Instead of an inductive coupling a hard wired connection may be established between interface 82 and battery charger 60 through a set of hard wired plugs (not shown).

For the purposes of this discussion it is now assumed that patient 32 suffers a cardiac attack. An untrained attendant notices that the patient 32 needs help. Depending on the physical location of the system and the patient 32, the attendant may be anyone in a hospital, a home or even a passerby. The attendant rushes to the pulse generator 12, removes it from the base station 14 and returns to the patient 32. When the pulse generator 12 is removed, or when the activation is otherwise activated, a signal is generated. In step 212 the activation is detected. In step 214 instructions are provided for the attendant for connecting the pulse generator to the patient 32, including instructions for placing the pulse electrodes 18 (including defibrillation pads, pacing electrodes, etc.) and the sensing electrodes 20 (which may include a set of standard ECG electrodes) on the patient's body. The instructions may be provided on the display 50, and/or orally through speaker 54 or may be printed on the housing 40.

While the attendant is positioning the electrodes on the patient, the pulse generator 12 sends a message through the base unit 14 to the central station 36 indicating that the pulse generator 12 has been activated (step 218). In step 220 the CPU 90 receives the message, identifies the system 10 and its location (either from the message, or from data stored in its memory 94) and generates an alarm message on display 92 for a clinician to indicate that the system 10 as been activated. An alarm may also be sounded through speaker 98.

In step 222 the clinician contacts the attendant and requests orally information about the patient, including the patient's age, sex, height, weight, any apparent medical conditions, etc. The clinician also checks with attendant whether the electrodes have been positioned. In step 224 the positioning of the electrodes is checked. More specifically, the measurement circuit 122 (FIG. 5) measures the impedance between the various sense and pace electrodes. Based on these measurements the system or the clinician may determine if the electrodes are positioned correctly (Step 226). If they are not, then in step 224 the clinician requests the attendant to reposition the electrodes until they are positioned properly. The attendant and the clinician speak to each other via the link established between the pulse generator 12 and station 36 through the speakers 54, 98 and microphones 56 and 100. While this conversation is going on the pulse generator 12 also sends data to the central station 36, including the impedance data collected by the measuring circuit 122. Based on these measurements a set of these electrodes are selected for acquiring the ECG signal. If the impedance from the sense electrodes is not satisfactory, the ECG signal for the patient may be acquired using the pulse electrodes or a combination of the pulse and sense electrodes.

Next in step 228, the selected electrodes are used to acquire an ECG signal indicative of the cardiac activity of the patient. The ECG signal is transmitted to the clinician for analysis (step 230). Alternatively the CPU 42 may be provided with a program for analyzing the ECG and to detect the R-waves for the patient. Once the R-waves are detected the CPU 42 can detect the current heart rate (HR) of the patient. This heart rate can also be sent to the central station in step 230.

Next, in step 232, the cardiac condition of the patient is classified. Preferably the criteria for this classification takes into consideration the current heart rate and other criteria such as the amplitude of the ECG or rate variability. Information from other optional sensors indicative of other parameters such as oxymetry (SpO2), patient impedance, motion sensing or blood pressure may also be used.

Once the cardiac condition has been categorized, an appropriate therapy can be selected in step 234. Steps 232 and 234 can be performed by the pulse generator 12 and/or the central station 36.

In step 236 the clinician informs the attendant that appropriate therapy is going to be administered to the patient. In step 238 the clinician initiates a command at his computer which is transmitted to the pulse generator 12. In step 240 the pulse generator 240 provides a final warning to the attendant of the imminent therapy. This step is especially important in case of high energy level defibrillation shocks since these shocks may injure the attendant.

Finally, in step 242 the pulse generator applies therapy to the patient in form of appropriate pulses, i.e., antibradicardia pacing pulses in case of bardycardia, antitachycardia pacing pulses in case of ventricular tachycardia, defibrillation shocks in case of ventricular fibrillation, and so on. Preferably, the therapy is delivered synchronously with the intrinsic heart beat of the patient as indicated by the detected R-waves.

After the therapy has been applied in step 242, the system returns to step 228 to acquire the new ECG for the patient, thereby determining if the therapy was effective or not.

If communication is lost, or never established between the base station 14 and central station 36, then a fallback mode may be provided in which the pulse generator generates pulses for the therapy on its own in an automatic mode and without instructions from the clinician. An external defibrillator of this type is described in commonly assigned co-pending application Ser. No. ______ incorporated herein by reference.

The system described in FIGS. 1-5 can be used in a home or an office. For home use, the system is advisable for survivors of SCA or a heart attack, for patients diagnosed with congestive heart failure, patients being monitored at home after cardiac surgery such as a cardiac bypass or valve replacement and patients associated with several risk factors which indicate possible heart attacks, the symptoms including high blood pressure, family history of cardiac problems or obesity.

Since large number of cardiac emergencies occur in office buildings, the system may also be installed there as well. However, since such buildings are relatively large as compared to private residences, a more robust system is envisioned for this implementation including stronger RF transceivers between the base station and the pulse generator.

In an alternate embodiment of the invention, the base station 14 is eliminated and the pulse generator is equipped with a transceiver or other means of accessing the central station, including a cellular telephone or other wireless means which are becoming quite popular. For this purpose the pulse generator 12 may be equipped with modem 57 which can facilitate communication directly with the central station 36.

In summary, a cardiac system is disclosed in which a relatively simple defibrillation device is provided which is operated/controlled from a remote location through a communication link. The defibrillation device may be provided as a stationary or a portable unit. Stationary units may include home-type version to be used by patients at home, or in a more robust office-type version for a clinician. In either case, the stationary device may include a base station which is mounted on a wall or other solid mounting position and a detachable member that can be dismounted from the base station and carried to the patient.

The portable unit preferably has a unitary construction including the defibrillation, control and communication circuitry. Communication from the portable unit may be achieved through a standard telephone, a cell telephone, satellite communication etc.

A large number of therapies may be applied through the device, including:

defibrillation, delivered by the device in response (preferably) to a command from a remote operator, with a bystander having the capability of overriding the remotely initiated shocks;

pacing controlled either locally or remotely;

drug delivery initiated either locally or remotely, including antiarrhythmic drugs or thrombolic drugs (for this purpose, the device may include a storage compartment and, if necessary, a syringe or other means of administering the same);

The system may also include one or more of the following options:

voice and/or video communication means between the remote operator and the bystander and/or patient;

event recorders (located locally or remotely) to document the symptoms and therapy applied to the patient and the efficacy of the system.

The defibrillation device described here has the following advantages:

it is simple and easy to use, especially by an untrained attendant;

it can be provided without any external controls thereby insuring that it is not mishandled;

it includes a base station which can be mounted on a wall and located in a visible and accessible site, and a pulse generator mounted at the base station;

the pulse generator has a speaker and microphone used for audio communication with a clinician at a central station;

the communication link is established by RF between the pulse generator and the base station and by standard telephone lines between the base station and the central station;

the device has self adhesive ECG monitoring and defibrillation electrodes attached to the patient by an attendant;

a set of 12 ECG sense electrodes are used to capture the ECG signals, these signals being used by the central station, or the pulse generator to diagnose the patient;

the pulse generator is capable of delivering antiarrhythmia therapy ranging from antibradycardia pacing pulses to high energy defibrillation shocks, the therapy being initiated by the central station;

it has an integral self-diagnosing circuitry which activates itself automatically or is randomly activated in demand from a remote location, i.e. the central substation; if a malfunction is detected the central station is automatically notified;

the results of the diagnosis are monitored by the central station;

data from a plurality of cardiac systems is stored in a central station;

the system has an alphanumeric display that can be used provide instructions to the attendant;

the system has rechargeable batteries that can be charged while the device us on standby to save power;

the system generally is operated from the remote central station which so that the attendant need not have special qualifications to operate the required devices.

Obviously numerous modifications may be made to this invention without departing from its scope as defined in the appended claims.

We claim:

1. An external cardiac device that can be used to apply therapy to any patient in accordance with instructions from a remote location, comprising:
an electrical coupler adapted to couple externally to the body of a patient;
a sense circuit coupled to said coupler to sense a physiological signal of the patient indicative of a cardiac condition of the patient;
a controller adapted to receive a command from the remote location;
a therapy delivery circuit adapted to deliver therapy to said patient to correct said cardiac condition in response to said command; and
a communication member coupling said controller to said remote location for receiving said command.

2. The device of claim 1 further comprising a pulse generator and base station, said pulse generator being removably mounted on said base station.

3. The device of claim 2 wherein said therapy delivery circuit and said sense circuit are disposed in said pulse generator.

4. The device of claim 2 wherein said communication member includes a communication link said pulse generator to said base when said pulse generator is removed therefrom, and a transceiver disposed in said base for establishing communication with the remote location.

5. The device of claim 1 wherein said communication member includes a transceiver for establishing communication with the remote location directly.

6. The device of claim 1 wherein said therapy delivery circuit is adapted to generate one of antitachycardia therapy, defibrillation therapy and antibradicardia therapy.

7. A publicly accessible external cardiac system for generating a therapy for a patient suffering an abnormal cardiac condition in response to commands from a clinician at a remote location, said system comprising:
a first electrode adapted to be attached to the patient;
a detector adapted to detect an electrical signal from the patient through said electrode;
a transceiver adapted to transmit an information signal to the clinician indicative of the patient's cardiac condition;
a controller coupled to said transceiver and adapted to receive a command from the clinician; and
a therapy generator coupled to said controller and adapted to generate a therapy selected to terminate said cardiac condition in response to said command.

8. The system of claim 7 further comprising a second electrode attached to said patient and being coupled to said therapy generator to deliver said therapy to the patient.

9. The system of claim 7 wherein said therapy generator is coupled to said detector and is adapted to generate said therapy to the patient's heart in synchronism with the signal from the patient's heart.

10. The system of claim 7 further comprising a pulse generator incorporating said first electrode, said detector, said controller and said therapy generator; and a base station incorporating said transceiver.

11. The system of claim 10 further comprising a communication link between said pulse generator and said base station to exchange signals with said transceiver.

12. The system of claim 10 wherein said pulse generator includes a rechargeable battery and a charging circuit and wherein said base station includes a power supply coupled to said charging circuit to provide power.

13. The system of claim 10 further comprising a speaker and a microphone to allow an operator to communicate with the clinician.

14. The system of claim 13 wherein said speaker and said microphone are associated with said pulse generator.

15. The system of claim 10 further comprising a display for showing messages to the attendant.

15. The system of claim 7 further comprising a diagnostic circuit adapted to run tests on said system to determine if said system is operational.

16. The system of claim 7 wherein said detector circuit is adapted to detect intrinsic cardiac signals and said controller is adapted to automatically generate said command in synchronism with said intrinsic cardiac signals.

17. The system of claim 7 wherein said controller is programmed to perform in a first mode wherein the controller is responsive to commands from the clinician and a second mode in which the controller activates said therapy generator automatically.

18. A method of providing cardiac therapy to a patient suffering from a cardiac condition using an external cardiac device, said method comprising the steps of:
attaching said device to the patient to sense an input signal indicative of intrinsic cardiac signals;
detecting an abnormal condition based on said input signal;
sending information regarding said patient to an operator at a location remote from said device;
receiving a command from the operator; and
applying therapy with said device in response to said command to correct said abnormal condition.

19. The method of claim 18 wherein said step of detecting includes detecting an ECG signal and detecting a heart rhythm based on said ECG signal.

20. The method of claim 18 further comprising performing an impedance measurement after said electrode is attached to said patient to determine if said external electrode is properly attached to the patient.

21. The method of claim 18 further comprising data logging each episode of cardiac condition and the corresponding therapy.

22. The method of claim 18 wherein said external defibrillator includes a display, further comprising providing on said display instructions for the operation of the defibrillator.

23. The method of claim 18 wherein said device includes a communication module, further comprising generating a message to a remote location indicative of the condition of the patient and sending said message to said remote location using said communication module.

24. The method of claim 18 wherein said device further comprises a speaker and a microphone further comprising providing instructions to an attendant regarding the attachment and operation of said device by the operator using said speaker and microphone.

25. The method of claim 18 wherein said external device includes another electrode and wherein said therapeutic pulses are applied to the patient through said another electrode.

26. The method of claim 18 wherein said step of applying therapy to the patient includes applying one of a antitachycardia therapy, defibrillation therapy and antibradicardia therapy.

27. The method of claim 18 wherein said device includes a pulse generator and a base station, further comprising generating an indication signal when said pulse generator and said base station are separated.

28. The method of claim 27 further comprising transmitting said indication signal to the operator to indicate that the device has been activated.
